(19) United States
(12) Patent Application Publication
(10) Pub. No.: US 2006/0247594 A1
(43) Pub. Date: Nov. 2, 2006

(54) Absorbent garments with graphic variety

(76) Inventors: Lisa L. Nickel, Menasha, WI (US); Kristi J. Bryant, Appleton, WI (US); Joseph P. Fell, Appleton, WI (US); Kellie M. Goodrich, Appleton, WI (US); Susan M. Oates, Appleton, WI (US)

Correspondence Address: KIMBERLY-CLARK WORLDWIDE, INC., 401 NORTH LAKE STREET, NEENAH, WI, 54956, US

(21) Appl. No.: 11/117,901

(22) Filed: Apr. 29, 2005

(51) Int. Cl. A61F 13/15

(52) U.S. Cl. 604/389; 604/391

(57) ABSTRACT

A variety of graphics on the absorbent article ears may provide several benefits. One benefit may be an increased interaction between the caregiver and the user. The graphic may become visible to the caregiver and user only after they have been fastened to the article. This provides a “surprise” aspect to which specific graphic may be on the ear.

### BACKGROUND OF THE INVENTION

Personal wear articles find widespread use as personal care products including, without limitation, diapers, children's toilet training pants, adult incontinence garments, sanitary napkins and the like. Certain such articles are generally considered to be disposable in that they are usually intended to be discarded after a limited period of use, i.e., the articles are not intended to be laundered or otherwise restored for reuse. Disposable absorbent articles typically comprise an absorbent body disposed between a liner, which contacts the wearer's skin, and an outer cover, which inhibits liquid body waste absorbed by the absorbent body from leaking out of the article. The liner of the absorbent article is typically liquid permeable to permit liquid body waste to pass through for absorption by the absorbent body.

Conventional absorbent articles also typically include some type of fastening system for securing the absorbent article in an assembled configuration and/or for fitting the article on the wearer, such as on the wearer's waist in the case of diapers and training pants. In many such applications, the fastening system is releasable and refastenable so that the article can be temporarily removed and then refastened to the wearer.

It is further known to apply a graphic, such as in the form of a character, object and/or alphanumeric (e.g., numbers, words, phrases, instructions, etc.) to personal wear articles to enhance the aesthetic or otherwise visual appearance or usefulness of the article. Graphics applied to such disposable articles may also provide visual assistance to the wearer or to a caregiver securing the article on the wearer.

Many caregivers and parents utilize the absorbent article changing process as a time to interact with the child. One of the interactions may be an inspection and discussion about the absorbent article. Many caregivers allow the child to play with the article before donning. Further the fasteners usually become visible to the child after the donning process. Traditional packages of absorbent articles may contain a single graphics on the fasteners or non at all. Therefore the child and caregiver may become bored with, or accustomed to, the graphic on the fasteners, removing most if not all expectation of viewing the fastener graphics.

In view of the aforementioned problems, there arises the need for absorbent articles having a variety of graphics.

### SUMMARY

In response to the foregoing need, the present inventors undertook intensive research and development efforts that resulted in the discovery of an absorbent article including a variety of graphics on the absorbent article ears. A variety of graphics on the absorbent article ears may provide several benefits. One benefit may be an increased interaction between the caregiver and the user. The graphic may become visible to the caregiver and user only after they have been fastened to the article. This provides a “surprise” aspect to which specific graphic may be on the ear. A second potential benefit results if the ear is a separately attached element. In this case the graphic may be printed on an element having a much shorter repeat length than the absorbent article; this may result in lower cost production, or a less complex process.

One version of the present invention includes a package of disposable absorbent articles including a first absorbent article and a second absorbent article. The first absorbent article defines opposed first and second fastener ears and comprises at least one fastener located on each fastener ear, a first graphic on the first fastener ear and a second fastener on the second fastener ear. The second absorbent article defines opposed first and second fastener ears and comprises at least one fastener located on each fastener ear, a third graphic on the first fastener ear and a fourth fastener on the second fastener ear. The first, second, third and fourth graphics include at least three different graphics.

Another version of the present invention provides a package of disposable absorbent articles including a first absorbent article and a second absorbent article. The first absorbent article defines a bodyfacing surface and a garment facing surface. The first absorbent article includes an absorbent core located between the bodyfacing surface and the garment facing surface and a fastening system. The fastening system including first and second fastener ears and including at least one fastener located on each fastener ear. A first graphic is included on the first fastener ear and a second graphic is included on the second fastener ear, each fastener being configured to engage at least a portion of the garment facing surface. The second absorbent article defines a bodyfacing surface and a garment facing surface. The second absorbent article including an absorbent core located between the bodyfacing surface and the garment facing surface and a fastening system. The fastening system including third and fourth fastener ears and including at least one fastener located on each fastener ear. A third graphic is included on the third fastener ear and a fourth graphic is included on the fourth fastener ear, each fastener being configured to engage at least a portion of the garment facing surface; wherein the first, second, third and fourth graphics comprise at least three different graphics.

Still another version of the present invention provides a package of disposable absorbent articles including a first absorbent article, a second absorbent article and a third absorbent article. The first absorbent article defines opposed first and second fastener ears and including at least one fastener located on each fastener ear. A first graphic included on the first fastener ear and a second graphic included on the second fastener ear. The second absorbent article defines opposed third and fourth fastener ears and including at least one fastener located on each fastener ear. A third graphic included on the third fastener ear and a fourth graphic included on the fourth fastener ear. The third absorbent article defines opposed fifth and sixth fastener ears and includes at least one fastener located on each fastener ear. A fifth graphic included on the fifth fastener ear and a sixth graphic included on the sixth fastener ear. The first, second, third, fourth, fifth and sixth graphics comprise at least six different graphics.

### DRAWINGS

The foregoing and other features, aspects and advantages of the present invention will become better understood with regard to the following description, appended claims and accompanying drawings where:

FIG. 1 illustrates a plan view of a disposable absorbent article in an unfolded, flat-out, uncontracted state (i.e., with all elastic induced gathering and contraction removed), with the bodyfacing surface of the article facing the viewer and with portions of the article partially cut away to illustrate underlying features;

FIG. 2 illustrates a second plan view of the disposable absorbent article of FIG. 1 in an unfolded, flat-out, uncontracted state, with the garment facing surface of the article facing the viewer and with portions of the article partially cut away to illustrate underlying features;

FIG. 3 illustrates a plan view of one version of an ear suitable for incorporation into a disposable absorbent article;

FIG. 4 illustrates a package of absorbent articles with portions of the package partially cut away to illustrate the disposable absorbent articles therein;

FIG. 5 illustrates a version of two ears suitable for incorporation into a disposable absorbent article; and

FIG. 6 illustrates a second version of two ears suitable for incorporation into a disposable absorbent article.

### DESCRIPTION

The present invention relates to disposable absorbent articles and packages of disposable absorbent articles. While the various versions of the present invention are described in terms of a disposable absorbent article such as an infant diaper, the invention is equally applicable to other disposable absorbent articles such as adult incontinence garments.

With regard to the designated surfaces of a disposable absorbent article and its components, the various upper or bodyfacing surfaces are configured to face toward the body of the wearer when the disposable absorbent article is worn by the wearer for ordinary use. The various opposing, lower or garment facing surfaces are configured to face away from the wearer's body when the disposable absorbent article is worn by the wearer.

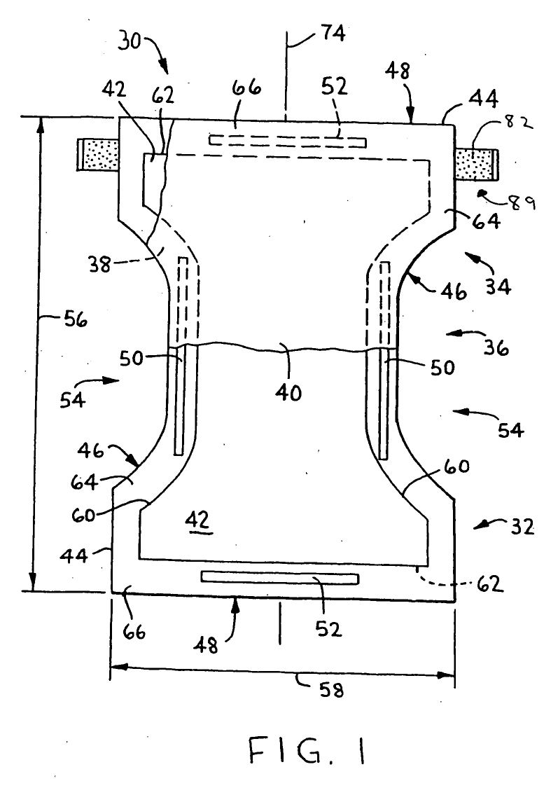

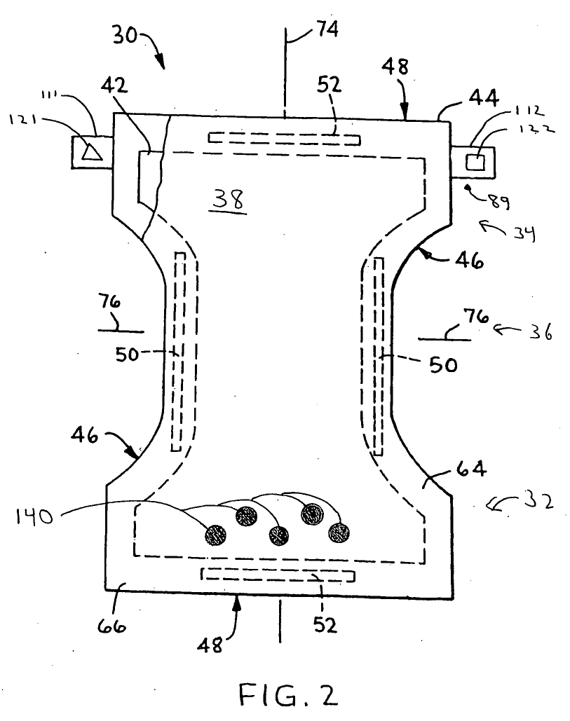

Referring now to the drawings, FIG. 1 illustrates a disposable absorbent article such as a disposable diaper (30) in an unfolded, flat-out, uncontracted state (i.e., with all elastic induced gathering and contraction removed). Portions of the structure are partially cut away to more clearly show the interior construction of the diaper (30), with the surface of the diaper (30) which contacts the wearer facing the viewer. FIGS. 1 and 2 illustrate a disposable diaper (30) as having a first region (32), a second region (34) and a crotch portion (36) located between the first and second regions. The diaper (30) includes a backsheet (38), a topsheet (40), and an absorbent core (42) situated between the backsheet and the topsheet. The outer edges of the diaper (30) define a periphery (44) with transversely opposed, longitudinally extending side edges (46); longitudinally opposed, transversely extending end edges (48); and a system of elastomeric gathering members, such as a system including leg elastics (50) and waist elastics (52). The longitudinal side edges (46) define the leg openings (54) for the diaper (30), and optionally, are curvilinear and contoured. The transverse end edges (48) are illustrated as straight, but optionally, may be curvilinear. The diaper (30) may also include additional components to assist in the acquisition, distribution and storage of bodily waste. For example, the diaper (30) may include a transport layer, such as described in U.S. Pat. No. 4,798,603, issued to Meyer et al., or a surge management layer, such as described in European Patent Application Publication No. 0 539 703, published May 5, 1993.

With regard to the designated surfaces of a disposable absorbent article and its components, the various upper or bodyfacing surfaces are configured to face toward the body of the wearer when the absorbent article is worn by the wearer for ordinary use. The various opposing or lower surfaces are configured to face away from the wearer's body when the absorbent article is worn by the wearer.

The diaper (30) generally defines a longitudinally extending length dimension (56), and a laterally extending width dimension (58), as representatively illustrated in FIG. 1. The diaper (30) may have any desired shape, such as rectangular, I-shaped, a generally hourglass shape, or a T-shape.

The backsheet (38) defines a length and a width which, in the illustrated version, coincide with the length and width of the diaper (30). The absorbent core (42) generally defines a length and width which are less than the length and width of the backsheet (38), respectively. Thus, marginal portions of the diaper (30), such as marginal sections of the backsheet (38), may extend past the transversely opposed, longitudinally extending terminal side edges (60) and/or the longitudinally opposed, transversely extending terminal end edges (62) of the absorbent core (42) to form side margins (64) and end margins (66) of the diaper (30). Alternatively, the marginal portions of the diaper (30), specifically the side margins (64) and the end margins (66), may extend inwardly from the periphery (44) of the diaper (30). The topsheet (40) is generally coextensive with the backsheet (38), but may optionally cover an area which is larger or smaller than the area of the backsheet, as desired. The backsheet (38) and topsheet (40) are intended to face the garment and body of the wearer, respectively, while in use. As used herein when describing the topsheet (40) in relation to the backsheet (38) and vice versa, the term “associated” encompasses configurations in which the topsheet is directly joined to the backsheet, and configurations where the topsheet is indirectly joined to the backsheet by affixing portions of the topsheet to intermediate members which in turn are affixed to at least portions of the backsheet. The topsheet (40) and the backsheet (38) can, for example, be joined to each other in at least a portion of the diaper periphery (44) by attachment mechanisms (not shown) such as adhesive bonds, sonic bonds, thermal bonds, pinning, stitching, or a variety of other attachment techniques known in the art, as well as combinations thereof.

The topsheet (40) suitably presents a bodyfacing surface which is compliant, soft feeling, and non-irritating to the wearer's skin. Further, the topsheet (40) may be less hydrophilic than the absorbent core (42), to present a relatively dry surface to the wearer, and is sufficiently porous to be liquid permeable, permitting liquid to readily penetrate through its thickness. A suitable topsheet (40) may be manufactured from a wide selection of web materials, such as porous foams, reticulated foams, apertured plastic films, natural fibers, synthetic fibers (for example, polyester or polypropylene fibers), or a combination of natural and synthetic fibers. The topsheet (40) is suitably employed to help isolate the wearer's skin from liquids held in the absorbent core (42).

Various woven and nonwoven fabrics may be used for the topsheet (40). For example, the topsheet (40) may be composed of a meltblown or spunbonded web of polyolefin fibers. The topsheet (40) may also be a bonded-carded web composed of natural and/or synthetic fibers. The topsheet (40) may be composed of a substantially hydrophobic material, and the hydrophobic material may, optionally, be treated with a surfactant, or otherwise processed, to impart a desired level of wettability and hydrophilicity. Specifically, the topsheet (40) may be a nonwoven, spunbond, polypropylene fabric composed of about 2.8 to about 3.2 denier fibers formed into a web having a basis weight of about 22 gsm and a density of about 0.06 g/cc.

The topsheet (40) may also be surface treated with about 0.3 weight percent of a surfactant mixture that contains a mixture of AHCOVEL Base N-62 surfactant and GLUCOPON 220UP surfactant in about a 3:1 ratio based on a total weight of the surfactant mixture. The AHCOVEL Base N-62 surfactant is purchased from Hodgson Textile Chemicals Inc., a business having offices in Mount Holly, N.C., and includes a blend of hydrogenated ethoxylated castor oil and sorbitan monooleate in a 55:45 weight ratio. The GLUCOPON 220UP surfactant is purchased from Henkel Corporation, Gulph Mills, Pa., and includes alkyl polyglycoside. The surfactant may also include additional ingredients such as aloe. The surfactant may be applied by any conventional means, such as spraying, printing, brush coating, foam or the like. The surfactant may be applied to the entire topsheet (40) or may be selectively applied to particular sections of the topsheet, such as the medial section along the longitudinal centerline of a diaper, to provide greater wettability of such sections.

The backsheet (38) may suitably be composed of a material which is either liquid permeable or liquid impermeable. It is generally desirable that the backsheet (38) be formed from a material which is substantially liquid impermeable. For example, a typical backsheet (38) can be manufactured from a thin plastic film or other flexible liquid impermeable material. Moreover, the backsheet (38) may be formed from a polyethylene film having a thickness of from about 0.012 mm (0.5 mil) to about 0.051 mm (2.0 mils). If desirous of presenting the backsheet (38) with a more cloth-like feel, the backsheet may include a polyethylene film having been laminated to the lower or opposing surface thereof a nonwoven web, such as a spunbond web of polyolefin fibers. For example, a polyethylene film having a thickness of about 0.015 mm (0.6 mil) may have thermally laminated thereto a spunbond web of polyolefin fibers, which fibers have a thickness of about 1.5 to about 2.5 denier per filament, which nonwoven web has a basis weight of about 24 gsm (0.7 osy). Methods of forming such cloth-like outer covers are known to those skilled in the art.

Further, the backsheet (38) may be formed of a woven or nonwoven fibrous web layer which has been totally or partially constructed or treated to impart a desired level of liquid impermeability to selected regions that are adjacent or proximate the absorbent core (42). Still further, the backsheet (38) may optionally be composed of micro-porous “breathable” material which permits vapors to escape from the absorbent core (42) while still preventing liquid exudates from passing through the backsheet.

The absorbent core (42) may include a matrix of hydrophilic fibers, such as a web of cellulosic fluff, mixed with particles of a high-absorbency material commonly known as superabsorbent material. In a particular version, the absorbent core (42) includes a mixture of superabsorbent hydrogel-forming particles and wood pulp fluff. The wood pulp fluff may be exchanged with synthetic polymeric, meltblown fibers or with a combination of meltblown fibers and natural fibers. The superabsorbent particles may be substantially homogeneously mixed with the hydrophilic fibers or may be non-uniformly mixed. The absorbent core (42) may be any suitable structure for absorbing and/or retaining exudates.

The absorbent core (42) may have any of a number of shapes. For example, the absorbent core (42) may be rectangular, I-shaped or T-shaped. It is often considered as desirable for the absorbent core (42) to be narrower in the crotch portion than the first or second region(s).

The high-absorbency material can be selected from natural, synthetic and modified natural polymers and materials. The high-absorbency materials can be inorganic materials, such as silica gels, or organic compounds, such as crosslinked polymers. The term “crosslinked” refers to any means for effectively rendering normally water-soluble materials substantially water insoluble, but swellable. Such means can include, for example, physical entanglement, crystalline domains, covalent bonds, ionic complexes and associations, hydrophilic associations, such as hydrogen bonding, and hydrophobic associations or Van der Waals forces.

Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent core include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthum gum, locust bean gum, and the like. Mixtures of natural and wholly or partially synthetic absorbent polymers can also be useful. Processes for preparing synthetic, absorbent gelling polymers are disclosed in U.S. Pat. No. 4,076,663, issued to Masuda et al., and U.S. Pat. No. 4,286,082, issued to Tsubakimoto et al.

The high-absorbency material may be in a variety of geometric forms. It is desired that the high-absorbency material be in the form of discrete particles. However, the high-absorbency material may also be in the form of fibers, flakes, rods, spheres, needles, or the like. Often, the high-absorbency material is present in the absorbent core (42) in an amount of from about 5 to about 100 weight percent based on total weight of the absorbent core.

The disposable absorbent articles described herein also include fasteners (82) for securing the absorbent article about the waist of the wearer. The illustrated versions of the diaper (30) include such fasteners (82). In at least one version, the fasteners (82) are situated in the second region (34) of the diaper (30), and located inboard each longitudinal extending side edge (46). The fasteners (82) may be configured to encircle the hips of the wearer and engage the backsheet (38) of the first region (32) of the diaper (30) for holding the diaper (30) on the wearer. Suitable fasteners are well known to those of skill in the art and can include adhesive tape tab fasteners, hook and loop fasteners, mushroom fasteners, snaps, pin, belts and the like, and combinations thereof. Desirably, the fasteners (82) are releasably engageable directly with the garment facing surface of the backsheet (38). Alternatively, the diaper (30) may include a fastening panel (not illustrated) situated in the first region (32) of the garment facing surface of the backsheet (38). In such a configuration, the fasteners (82) are releasably engageable with the fastening panel to maintain the diaper (30) about the waist of the wearer. As representatively illustrated in FIGS. 1-3, the fasteners (82) may be hook type fasteners and the backsheet (38) may be configured to function as a complimentary loop type fastener. Desirably, the fasteners (82) are hook type fasteners which are releasably engageable with the backsheet (38). Such an arrangement provides the ability to vary the size of the waist opening in very small increments over a wide range to fit the waist of the wearer. The fasteners (82) may have a variety of shapes and sizes which provide the desired fastening of the diaper (30) about the waist of the wearer.

The term “inboard” is intended to refer to the direction from a periphery or an edge toward a respective centerline, such as the transverse centerline (76) or the longitudinal centerline (74). The term “outboard” is intended to refer to a direction away from a respective centerline.

The diaper (30) may include ears (89). In particular arrangements, each ear (89) extends laterally at the opposed, lateral ends of at least one waist region of the backsheet (38), to provide terminal side sections of the absorbent article. In addition, each ear (89) may substantially span from a laterally extending, terminal waistband edge to approximately the location of its associated and corresponding leg opening section of the diaper.

In the various configurations of the invention, the ears (89) may be integrally formed with a selected diaper component. For example, ears (89) can be integrally formed from the layer of material which provides the backsheet (38), or may be integrally formed from the material employed to provide the topsheet (40). In alternative configurations, the ears (89) may be provided by one or more separately provided members that are connected and assembled to the backsheet (38), to the topsheet (40), in between the backsheet and topsheet, or in various fixedly attached combinations of such assemblies.

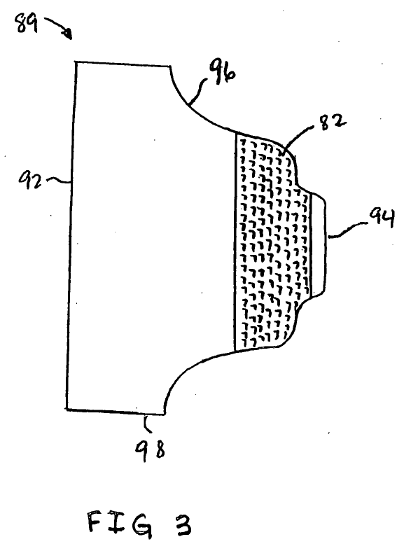

As illustrated in FIG. 3, the ear (89) may be provided by a separately provided member that includes a proximal edge (92), an opposed distal edge (94), a first connecting edge (96), and a second connecting edge (98). As used herein, the proximal edge (92) is that edge of the ear (89) which is connected or otherwise associated in an operable manner to a longitudinal extending side edge (46) of the diaper (30). The distal edge (94) is that edge of the ear (89) which is opposite the proximal edge (92) moving in a direction outboard from a longitudinal centerline (74) of the diaper (30). The first (96) and second (98) connecting edges connect the proximal edge (92) and the distal edge (94) thereby defining a body of material which at least partially defines an ear (89). The ear (89) may be made of a variety of materials including those that are extensible, elastomeric and/or non-elastomeric. As used herein when describing the diaper (30) in relation to the ear (89) and vice versa, the term “associated” encompasses configurations in which the diaper (30) is directly joined to the ear (89), and configurations wherein the diaper (30) is indirectly joined to the ear (89) by affixing the diaper (30) to intermediate members which in turn are affixed to the ear (89). As used herein, the ear (89) forms at least a portion of the side margin (64).

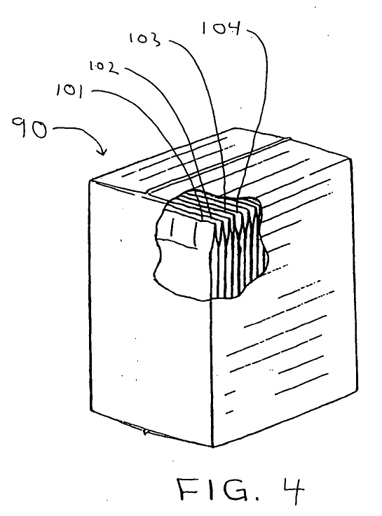

The present invention provides a package (90) including a plurality of the disposable absorbent articles described above, as illustrated in FIG. 4. The package (90) includes a first absorbent article (101) and a second absorbent article (102). The package may include a third absorbent article (103), a fourth absorbent article (104), or more absorbent articles. The first (101), second (102), third (103) and fourth (104) absorbent articles may all have a similar construction, for example a substantially identical absorbent capacity, alternatively, they may have two or more different constructions, for example, two or more different absorbent capacities.

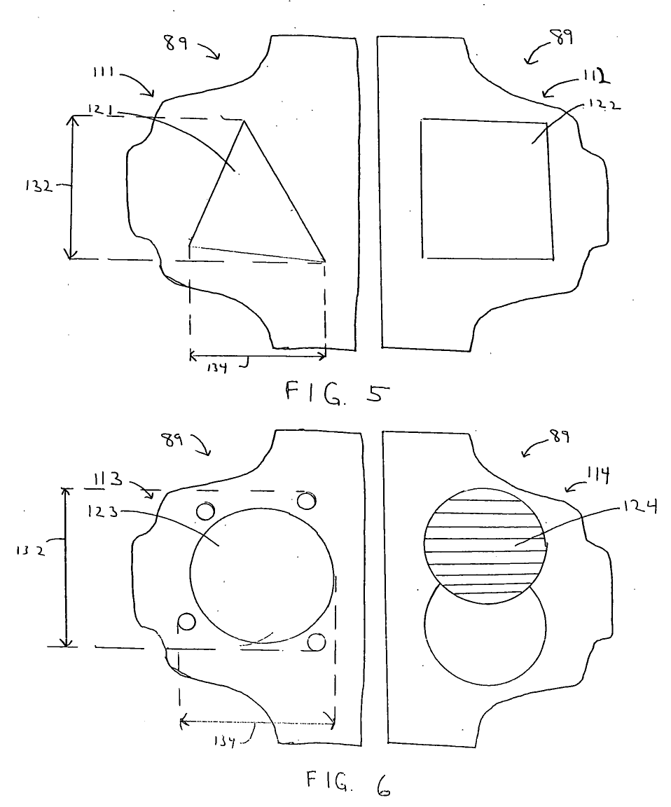

The first absorbent article (101) includes at least two fasteners (82) located on fastener ears (89) defining a first fastener ear (111) and a second fastener ear (112). The first (111) and second (112) fastener ears are separately illustrated in FIG. 5. The second absorbent article (102) also includes at least two fasteners (82) located on fastener ears (89) defining a third fastener ear (113) and a fourth fastener ear (114). The third (113) and fourth (114) fastener ears are separately illustrated in FIG. 6. The third absorbent article also includes at least two fasteners (82) located on fastener ears (89) defining a fifth fastener ear and a sixth fastener ear.

The first fastener ear (111) has a first graphic (121). The first graphic (121) may be located on either the garment facing surface or the bodyfacing surface of the first fastener ear (111). The first graphic (121) may include a plurality of graphics. Alternatively, as representatively illustrated in FIG. 5, the first graphic (121) may include a single graphic. The first graphic (121) may include, but are not limited to, scenes, characters, animals, objects, alphanumerics such as numbers, letters, words, phrases and the like. In particular aspects, the first graphic (121) may also be gender specific; that is, the first graphic (121) may be a graphic that may be generally considered to be of interest to boys or to girls.

The second fastener ear (112) has a second graphic (122), the third fastener ear (113) has a third graphic (123), the fourth fastener ear (114) has a fourth graphic (124), the fifth fastener ear has a fifth graphic, and the sixth fastener ear has a sixth graphic. Any graphic suitable for the first graphic (121) is also suitable for the second (122), third (123), fourth (124), fifth, or sixth graphic.

The first (121), second (122), third (123) and fourth (124) graphics comprise at least three different graphics. The first (121), second (122), third (123) and fourth (124) graphics may comprise four different graphics. Further, the first (121), second (122), third (123), fourth (124), fifth and sixth graphics may comprise five or six different graphics. For purposes of the present invention, graphics are considered to be different when they differ in size, shape, color or pattern. As shown in FIGS. 5 and 6, the first graphic (121) is a triangle, the second graphic (122) is a rectangle, the third graphic (123) is a number of circles and the fourth graphic (124) is a stacked pair of circles with the top circle including a pattern.

Moreover, each graphic (121, 122, 123 or 124) may define a total graphic area. The total graphic area may be calculated by multiplying the largest dimension of the graphic (121, 122, 123 or 124) in the longitudinal direction (the graphic length, indicated at 132) by the largest dimension of the graphic (121, 122, 123 or 124) in the lateral direction (the graphic width, indicated at 134) (FIGS. 5, 6). Therefore, in one aspect, the graphic area may be at least 9 square cm. In another aspect, the graphic area may be at least 16 square cm. Alternatively, as mentioned above, the fastener ear (89) may include a plurality of graphics (121, 122, 123 or 124). Thus, the plurality of graphics (121, 122, 123 or 124) may, in total, define a total graphic area. The total graphic area may be calculated by multiplying the graphic length of the plurality of interior graphics in the longitudinal direction, by the interior graphic width of the plurality of graphics in the lateral direction. The plurality of graphics may define a total graphic area of at least 4 square cm. Such total graphic areas as described above suitably draw the attention of the wearer to the graphics.

The first (101), second (102), third (103) and fourth (104) absorbent articles of the various aspects of the present invention may further include at least one graphic (140) disposed on the garment facing surface. Suitably, the diaper (30) may include a plurality of graphics (140) on the garment facing surface, as illustrated in FIG. 2. The graphics (140) may include, but are not limited to, scenes, characters, animals, objects, alphanumerics such as numbers, letters, words, phrases and the like; highlighting or emphasizing the leg openings (54) in order to make product shaping more evident or visible to the user; highlighting or emphasizing areas of the product to simulate functional components such as elastic leg bands, elastic waistbands, simulated “fly openings” for boys, ruffles for girls; highlighting areas of the product to change the appearance of the size of the product; registering wetness indicators, temperature indicators, and the like in the product; registering a back label, or a front label, in the product; and registering written instructions at a desired location in the product.

The graphics (140) can be formed on or applied to the backsheet (38), the fastening panel or another substrate bonded to or placed with or placed near the backsheet (38) by any suitable technique. The graphics (140) may be in the first waist region (32), the crotch region (36) or the second waist region (34). The graphics (140) may be registered with other components of the absorbent article; alternatively, the graphics (140) may be non-registered. For purposed of the present invention, the term registered means that element in a repeating pattern are place in the same location on a first (101) and second (102) absorbent article. For purposed of the present invention, the term non-registered means that element in a repeating pattern are not place in the same location on a first (101) and second (102) absorbent article.

The graphics (121, 122, 123, 124, 140) of the present invention may also be configured to define a graphic theme. It should be noted that in order to establish a graphic theme, not every graphic (121, 122, 123, 124, 140) need be directly related to the graphic theme, however, it can be appreciated that to effectively establish a graphic theme, it is desirable to have at least half of the graphics (121, 122, 123, 124, 140) contribute to defining the graphic theme, more desirably the majority of the graphics (121, 122, 123, 124, 140) contribute to defining the graphic theme, and still more desirably substantially all of the graphics (121, 122, 123, 124, 140) contribute to defining the graphic theme. Similarly, it can be appreciated that where certain graphics (121, 122, 123, 124, 140) are not contributing to defining the theme of the other graphics (121, 122, 123, 124, 140), it can be most effective to at least have the non-contributing graphics (121, 122, 123, 124, 140) be neutral or not in opposition toward the graphic theme.

The graphics (121, 122, 123, 124, 140) may define a graphic theme when the subject matter of one graphic (121, 122, 123, 124, 140) is the same as or is associated with the subject matter of another graphic (121, 122, 123, 124, 140). For example, the graphics (121, 122, 123, 124, 140) may be related by a unifying subject or common story line, which could be generally known through books, movies, common children's activities, or other sources to provide a graphic theme. By way of example, two objects are considered the same as or associated in subject matter where the images are identical; separately illustrate different sizes, shapes, colors of a common object; each illustrate one and the other of two objects that are commonly associated with one another, such as the moon and stars, a body of water and water toys, a sandbox and suitable toys, a baseball bat and ball, a barn and animals, or the like; illustrate different items used in a particular activity, such as a sporting activity, a gardening activity or the like; jointly illustrate geometrically mating or engaging elements such as a triangle and a triangularly-shaped aperture, or two halves of a zipper; each illustrate one part of a multipart picture; or the like. Similarly, two text messages are considered related in subject matter where the messages: are identical; jointly form a sentence, thought, or action such as “jump” and “up”; each refer to one and the other of two items that are commonly associated with one another, such as “bat” and “ball,” “Big” and “Kid,” “Big” and “Girl,” or “Big” and “Boy”; jointly present a question and answer; or the like. Likewise, a text message and a pictorial image are considered to be related in subject matter where the text names, defines or describes the image; or the like.

Conversely, and by way of illustration and without wishing to be limited to the enumerated examples, two objects are considered unrelated in subject matter where the images: illustrate items that are neither identical nor illustrate two objects that are not commonly associated with one another, such as an animal and a building block, a jump rope and a flower, a car and a star, a letter of the alphabet and a water toy, a fish and an apple, illustrate items used in unrelated activities, such as items used in sporting activities and items used in gardening activities, or other unrelated activities, or the like. Similarly, two text messages are considered unrelated in subject matter where the messages are neither identical nor jointly form a sentence, thought, or action; refer to two items that are not commonly associated with one another, such as “ball” and “flower,” “fish” and “pencil,” “car” and “ghost,” or other such unrelated words, or the like. Likewise, a text message and a pictorial image are considered to be unrelated in subject matter where the text does not name, define, describe or otherwise relate to the image.

Thus, the ear (89) graphics (121, 122, 123, 124) may be related or unrelated to a graphic theme that may be defined by the graphics (140) on the garment facing surface. In particular, and without wishing to be limited to the specific embodiments listed, suitable examples may include the graphics (140) on the garment facing surface being a racquet, bat, glove, other sporting equipment or the like and the ear (89) graphic (121, 122, 123, 124) comprising balls, or being related sporting equipment or the like; the graphics (140) being a butterfly net or the like and the ear (89) graphics (121, 122, 123, 124) comprising butterflies or the like; the graphics (140) being a fish, a boat or the like and the ear (89) graphics (121, 122, 123, 124) being a shell, water toys or the like; the graphics (140) being flowers, plants, gardening tools or the like and the ear (89) graphics (121, 122, 123, 124) comprising flowers or plants; the graphics (140) being a specific environment such as a barn, silo, tractor or the like and the ear (89) graphics (121, 122, 123, 124) comprising cows, chickens, sheep, or the like which are specifically adapted to the environment; the graphic (140) being a telescope, stars, planets or the like and the ear (89) graphics (121, 122, 123, 124) being rockets, spaceships or flying saucers.

In aspects where the ear (89) graphic (121, 122, 123, 124) is related to a theme established by the graphics (140), it provides an opportunity for the wearer and the caregiver to interact and can improve the donning experience.

As mentioned above, the graphics (140) on the garment facing surface and the ear (89) graphics (121, 122, 123, 124) may be disposed on the absorbent article (30) using a variety of methods. For example, the graphics (140, 121, 122, 123, 124) may suitably be disposed on the absorbent article (30) by being imprinted thereon using a flexographic printing process. Flexographic printing is a conventional printing technique which uses flexible, raised rubber or photopolymer plates to carry an inked image to a substrate, such as a backsheet (38), fastening panel, or an ear (89). As an example, flexographic printing apparatus are shown and/or described in U.S. Pat. No. 5,458,590 (Schleinz et al.); U.S. Pat. No. 5,566,616 (Schleinz et al.); U.S. 2003/0019374A1 (Harte); and U.S. Pat. No. 4,896,600 (Rogge et al.). The first (101) and second (102) absorbent articles may have graphics (121, 122, 123, 124, 140) that are related, for example the first absorbent article (101) may have graphics (121, 122, 123, 124, 140) including a baseball theme, and the second absorbent article (102) may have graphics (121, 122, 123, 124, 140) including a second baseball theme. Alternatively, the first (101) and second (102) absorbent articles may have graphics (121, 122, 123, 124, 140) that are unrelated, for example the first absorbent article (101) may include a baseball theme, and the second absorbent article (102) may include a construction theme. Further the ear (89) graphics and graphics (140) on the garment facing surface in the first waist region (32) on the third (103) and fourth (104) absorbent articles may be related or unrelated to the graphics (121, 122, 123, 124, 140) of the other absorbent articles.

As various changes could be made in the above constructions and methods, without departing from the scope of the invention, it is intended that all matter contained in the above description and shown in the accompanying drawings shall be interpreted as illustrative and not in a limiting sense.

When introducing elements of the invention or the preferred aspect(s) thereof, the articles “a”, “an”, “the” and “said” are intended to mean that there are one or more of the elements. The terms “comprising”, “including” and “having” are intended to be inclusive and mean that there may be additional elements other than the listed element.

What is claimed is:

1. A package of disposable absorbent articles comprising a first absorbent article and a second absorbent article, the first absorbent article defines opposed first and second fastener ears and comprises at least one fastener located on each fastener ear, a first graphic on the first fastener ear and a second fastener on the second fastener ear, the second absorbent article defines opposed first and second fastener ears and comprises at least one fastener located on each fastener ear, a third graphic on the first fastener ear and a fourth fastener on the second fastener ear, wherein the first, second, third and fourth graphics comprise at least three different graphics.

2. The package of claim 1 wherein the first, second, third and fourth graphics each comprises a single graphic.

3. The package of claim 1 wherein the first, second, third and fourth graphics comprise at least four different graphics.

4. The package of claim 1 wherein the first absorbent article has a first region, a second region, and a crotch region which extends between and connects the first region and the second region, the two fastener ears being situated in the second region, and graphics located in the first region; and the second absorbent article has a first region, a second region, and a crotch region which extends between and connects the first region and the second region, the two fastener ears being situated in the second region, and graphics located in the first region.

5. The package of claim 4 wherein the graphics in the first waist region on the first absorbent article and the graphics in the first region on the second absorbent article are substantially identical.

6. The package of claim 5 wherein the graphics in the first region on the first absorbent article and the graphics in the first region on the second absorbent article are non-registered graphics.

7. The package of claim 5 wherein the graphics on the garment facing surface in the first region on the first absorbent article and the graphics on the garment facing surface in the first region on the second absorbent article are registered graphics.

8. The package of claim 1 wherein the first graphic defines a first total graphic design area, the second graphic design defines a second total graphic design area, the third graphic design defines a third total graphic design area the fourth graphic design defines a fourth total graphic design area, the first, second, third and fourth total graphic design areas each being at least 4 square cm.

9. The package of claim 1 wherein the first absorbent article and the second absorbent have substantially identical absorbent capacities.

10. A package of disposable absorbent articles comprising a first absorbent article and a second absorbent article,
the first absorbent article defining a bodyfacing surface and a garment facing surface and comprising:
an absorbent core located between the bodyfacing surface and the garment facing surface; and
a fastening system, the fastening system comprising:
first and second fastener ears and comprises at least one fastener located on each fastener ear, a first graphic on the first fastener ear and a second graphic on the second fastener ear, each fastener being configured to engage at least a portion of the garment facing surface;
the second absorbent article defining a bodyfacing surface and a garment facing surface and comprising:
an absorbent core located between the bodyfacing surface and the garment facing surface; and
a fastening system, the fastening system comprising:
third and fourth fastener ears and comprises at least one fastener located on each fastener ear, a third graphic on the third fastener ear and a fourth graphic on the fourth fastener ear, each fastener being configured to engage at least a portion of the garment facing surface; wherein the first, second, third and fourth graphics comprise at least three different graphics.

11. The package of claim 10 wherein the first, second, third and fourth graphics each comprises a single graphic.

12. The package of claim 10 wherein the first, second, third and fourth graphics each comprises at least four different graphics.

13. The package of claim 10 wherein the first absorbent article has a first region, a second region, and a crotch region which extends between and connects the first region and the second region, the first and second fastener ears being situated in the second region, and graphics located in the first region; and the second absorbent article has a first region, a second region, and a crotch region which extends between and connects the first region and the second region, the third and fourth fastener ears being situated in the second region, and graphics located in the first region.

14. The package of claim 13 wherein the graphics on the garment facing surface in the first region on the first absorbent article and the graphics on the garment facing surface in the first region on the second absorbent article are substantially identical.

15. The package of claim 14 wherein the graphics on the garment facing surface in the first region on the first absorbent article and the graphics on the garment facing surface in the first region on the second absorbent article are non-registered graphics.

16. The package of claim 14 wherein the graphics on the garment facing surface in the first region on the first absorbent article and the graphics on the garment facing surface in the first region on the second absorbent article are registered graphics.

17. The package of claim 10 wherein the first graphic design defines a first total graphic design area, the second graphic design defines a second total graphic design area, the third graphic design defines a third total graphic design area the fourth graphic design defines a fourth total graphic design area, the first second, third and fourth total graphic design areas each being at least 4 square cm.

18. The package of claim 10 wherein the first absorbent article and the second absorbent have substantially identical absorbent capacities.

19. A package of disposable absorbent articles comprising a first absorbent article, a second absorbent article and a third absorbent article, the first absorbent article defines opposed first and second fastener ears and comprises at least one fastener located on each fastener ear, a first graphic on the first fastener ear and a second graphic on the second fastener ear, the second absorbent article defines opposed third and fourth fastener ears and comprises at least one fastener located on each fastener ear, a third graphic on the third fastener ear and a fourth graphic on the fourth fastener ear, the third absorbent article defines opposed fifth and sixth fastener ears and comprises at least one fastener located on each fastener ear, a fifth graphic on the fifth fastener ear and a sixth graphic on the sixth fastener ear, wherein the first, second, third, fourth, fifth and sixth graphics comprise at least six different graphics.
